(19) United States
(12) Patent Application Publication
(10) Pub. No.: US 2021/0299369 A1
(43) Pub. Date: Sep. 30, 2021

(54) Smelling Salt Inhaler

(71) Applicant: Jackson Jesionowski, Tucson, AZ (US)

(72) Inventor: Jackson Jesionowski, Tucson, AZ (US)

(21) Appl. No.: 16/830,299

(22) Filed: Mar. 26, 2020

(51) Int. Cl. A61M 15/08; A61M 21/00

(52) U.S. Cl. CPC A61M 15/08; A61M 2021/0016; A61M 21/00

(57) ABSTRACT

This present invention relates to a pocket inhaler containing smelling salts aka ammonia inhalants, and so constructed that when in use, it may be closed tight and no odour can escape, nor can the contents waste by evaporation.

### TECHNICAL INVENTION

The object of the present invention is to provide pocket inhaler containing smelling salts within an inhaler stick for use with the nostril.

### BACKGROUND

### Summary

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is the principal object of this invention to provide an inhaler which does not require an extraneous member for closing same when not in use

Another object of this, invention, is the provision of an inhaler which embodies, means for varying the opening to the inhaler whereby the flow through the inhaler may be varied as to quantity depending upon the contact within the nostrils.

Another object of the invention is the provision of an inhaler formed in the main of a plastic with which metal parts cooperate to avoid the inherent difficulty of operation of the parts due to the medicine attacking the same when all are made of plastic.

It is also an object of this invention to provide a smelling salts nasal inhaler which is attractive and pleasing, yet economical to manufacture and produce; whereby the inhaler is economical to purchase.

This Summary is provided merely for purposes of summarizing some example embodiments, to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is an exploded perspective view illustrating one preferred form for a salt nasal inhalation system embodying the novel features of the invention;

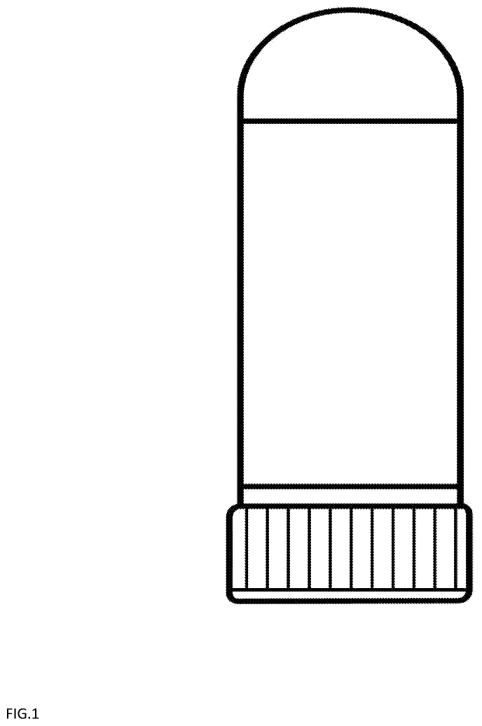

FIG. 2 is a front end elevation view taken generally of FIG. 1;

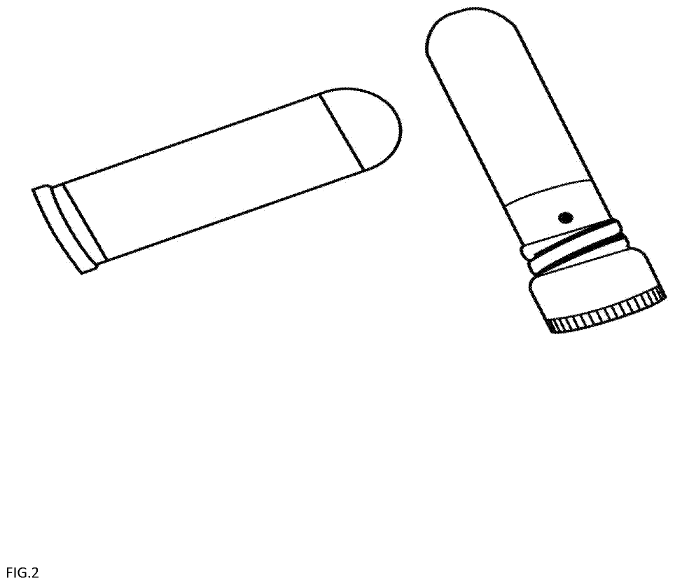

FIG. 3 is a horizontal view through a portion of the inhalation system

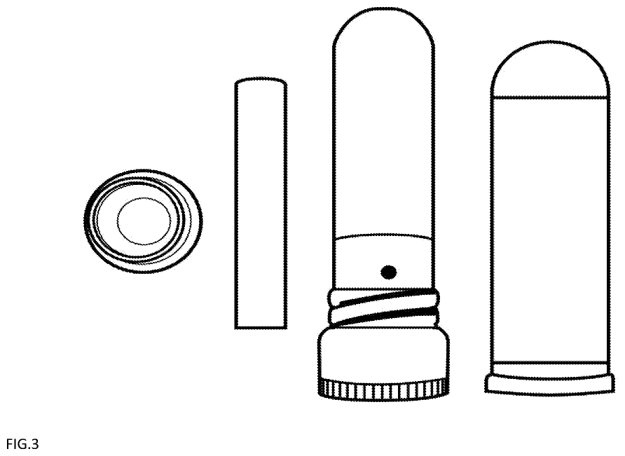

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The present invention includes an outer shell having one end rounded, as shown at, and provided an inhaler stick for use with the nostril through which the contents are inhaled. The inner stick of the container is provided with one or more perforations, which are out of registry with the passageway.

The container is provided near its outer end with a lug, which rides in an inclined slot in the wall of the shell, and flange, preferably knurled, is formed on the periphery of the container outside of the shell.

The inner stick is closed by a covering cap, which fits snugly and is practically air-tight so that the draft through the container passes inwardly through the openings, which are located inst above the knurled flange and are uncovered whenever the lug is moved to the lower end of the slot, but when the lug is loved into the notch, the holes are covered by the lower end of the shell and the holes are also covered by the plug So that the draft is cut off from both sides of the tablets or smelling salts within the container.

To support the tablets above the bottom of the container, so that they will be in the direct line of the draft through the same have provided an inside cap, which is provided with a plurality of perforations to permit a free draft which is ammonia-based smelling salts* are most commonly a mixture of two very simple compounds: Na2CO3 (sodium carbonate) and NH4Cl (ammonium chloride), therethrough. Alternatively, it would be ammonia hydroxide. This cap fits snugly within the container and is preferably provided with a slot in its side which engages with an inwardly projecting lug, to guide the cap within the container and form an abutment against which it may rest.

From the foregoing description, it will be apparent that the device may be easily manipulated to open or close the draft, that the tablets are supported in a position to produce a maximum effect, and that when the device is closed it is tight and there is no waste of the contents very simple compounds: Na2CO3 (sodium carbonate) and NH4Cl (ammonium chloride) or ammonia hydroxide.

The Abstract of the Disclosure is provided to allow the reader to quickly ascertain the nature of the technical disclosure. It is submitted with the understanding that it will not be used to interpret or limit the scope or meaning of the claims. Also, in the foregoing Detailed Description, it can be seen that various features are grouped in various embodiments to streamline the disclosure. This method of disclosure is not to be interpreted as reflecting an intention that the claimed embodiments require more features than are expressly recited in each claim. Rather, as the following claims reflect, the inventive subject matter lies in less than all features of a single disclosed embodiment. Thus the following claims are hereby incorporated into the Detailed Description, with each claim standing on its own as a separately claimed subject matter.

What is claimed is:

1. In a pocket inhaler, the combination of a shell having one end perforated and the other end open, a container telescopically received in the open end of said shell and having its outer end closed by a removable outer cap, a perforated inner cap fitting within the container and spaced from the outer cap and defining an inner cavity for containing a very simple compound: Na2CO3 (sodium carbonate) and NH4Cl (ammonium chloride) or ammonia hydroxide, the inner cap having a slot in its side, an internal lug on the container engaging the slot to limit the inward movement of the inner cap, means on the inner cap engageable by the outer cap for retaining the inner cap in position, and adjustable means for optionally establishing a draft longitudinally through the inner cap and the inner cavity and the perforated end of the shell.

2. According to claim 1, the present invention is a multi-use smelling salt inhaler
